(19) United States
(12) Patent Application Publication
(10) Pub. No.: US 2026/0101938 A1
(43) Pub. Date: Apr. 16, 2026

(54) ERGONOMIC MAGNETIC THERMAL GARMENT

(71) Applicant: Air Force Medical University, Xi'an (CN)

(72) Inventors: Bo GAO, Xi'an (CN); Dong WANG, Xi'an (CN); Zhuojing LUO, Xi'an (CN); Zhengxu YE, Xi'an (CN); Ming YAN, Xi'an (CN); Rundong CHAI, Xi'an (CN); Guanyi WANG, Xi'an (CN); Wei DUAN, Xi'an (CN)

(73) Assignee: Air Force Medical University, Xi'an (CN)

(21) Appl. No.: 19/337,971

(22) Filed: Sep. 24, 2025

(30) Foreign Application Priority Data
Oct. 12, 2024 (CN) 202411422859.1

(51) Int. Cl. A41D 1/00; A41D 31/06; A44B 18/00

(52) U.S. Cl. CPC A41D 1/002; A41D 31/06; A44B 18/00; A61N 2/00; A41D 2600/00

(57) ABSTRACT

The provided is an ergonomic magnetic thermal garment. An elastic adjustment piece is arranged at a middle opening of a jacket to adapt to different body shapes; both the jacket and a pair of pants are provided with magnetic thermal devices. The magnetic thermal devices include a magnetic control mechanism, an electric heating mechanism, and a control mechanism. The electric heating mechanism is arranged on an inner lining of the jacket and the pants and is connected to the control mechanism. The magnetic control mechanism is arranged on an outer lining of the jacket and the pants and is connected to the control mechanism. The control mechanism is also connected to an external power supply mechanism for providing a power supply. A magnetic thermal garment using a magnetic thermal treatment method is designed, so as to provide magnetic thermal intervention of varying durations and intensities to damaged tissue.

### CROSS-REFERENCE TO THE RELATED APPLICATIONS

This application is based upon and claims priority to Chinese Patent Application No. 202411422859.1, filed on Oct. 12, 2024, the entire contents of which are incorporated herein by reference.

### TECHNICAL FIELD

The present invention belongs to the technical field of medical care, and specifically discloses an ergonomic magnetic thermal garment.

### BACKGROUND

Magnetic thermal treatment is an effective treatment for musculoskeletal injuries. Existing treatments for musculoskeletal injuries are mainly bedside treatment devices, which are limited in treatment scenarios and require patients to receive treatment in medical facilities. Wearable treatment devices have to some extent solved the site limitations of magnetic thermal treatment, making home rehabilitation treatment possible.

Current wearable magnetic thermal treatment devices, such as magnetic thermal cervical vertebrae treatment devices and lumbar vertebrae treatment devices, have a small treatment range, limited location, and a single function, and cannot achieve integrated treatment.

### SUMMARY

To solve the foregoing problems, the present invention adopts the following technical solution.

An ergonomic magnetic thermal garment includes a jacket and a pair of pants, wherein an elastic adjustment piece is arranged at a middle opening of the jacket to adapt to different body shapes;
both the jacket and the pants are provided with magnetic thermal devices; the magnetic thermal devices include a magnetic control mechanism, an electric heating mechanism, and a control mechanism;
the electric heating mechanism is arranged on an inner lining of the jacket and the pants and is connected to the control mechanism;
the magnetic control mechanism is arranged on an outer lining of the jacket and the pants and is connected to the control mechanism;
the control mechanism is also connected to an external power supply mechanism for providing a power supply; and
neck, shoulders, back, chest, abdomen, waist, upper arms and forearms of the jacket, as well as thighs, calves and knees of the pants, are all detachably connected with fixing accessories for providing mechanical support.

Further, the electric heating mechanism includes electric heating plates, the electric heating plates are arranged on the inner linings of the jacket and the pants and connected to the control mechanism.

Further, the electric heating plate is composed of a plurality of small electric heating plates connected in parallel through a first flexible wire; and the plurality of electric heating plates are distributed on the neck, shoulders, back, chest, abdomen, waist, upper arms and forearms of the jacket, as well as the thighs, calves, knees and buttocks of the pants.

Further, a temperature of the small electric heating plate is 40-55° C.

Further, the magnetic control mechanism includes a plurality of coil assemblies, the coil assemblies are arranged on neck, shoulders, back, chest, abdomen, waist, upper arms and forearms of the jacket, as well as an outer lining of thighs, calves, knees and buttocks of the pants, and are connected in parallel through a second flexible wire, the coil assemblies are connected to the control mechanism and generate an electromagnetic field through the control mechanism.

Further, the control mechanism regulates a release position and pulse parameters of the electromagnetic field; and the electromagnetic field is composed of a pulse waveform with a frequency of 15 Hz.

Further, the jacket and the pants are designed according to curves of a human body.

Further, an inner lining of the jacket and the pants are made of a heat-resistant insulating material.

Further, the elastic adjustment piece is a hook-and-loop fastener, one of hook or loop portions of the hook-and-loop fastener is located on an outer surface of the garment, a portion matched to the hook or loop portions is located on an inner lining of the garment, a size of the hook-and-loop fastener located on the outer surface of the garment is larger than a size of the hook-and-loop fastener located on the inner lining of the garment.

### Beneficial Effects

The magnetic thermal garment is designed using the magnetic-thermal treatment method for magnetic-thermal intervention on the damaged tissues with different durations and different intensities. The ergonomic design enables the magnetic thermal garment to provide effective mechanical support for local tissues (such as the lumbar spine, cervical spine, shoulders, and knee joints), helping to reduce local injuries and enhance the treatment effect.

### BRIEF DESCRIPTION OF THE DRAWINGS

FIG. 1 is a schematic structural diagram of a jacket of an ergonomic magnetic thermal garment according to the present invention;

FIG. 2 is a schematic diagram of an internal structure of a jacket of an ergonomic magnetic thermal garment according to the present invention; and

FIG. 3 is a schematic diagram of an internal structure of pant of an ergonomic magnetic thermal garment according to the present invention.

Reference numerals: 1. jacket; 2. pants; 3. thighs; 4. calves; 5. upper arms; 6. forearms; 7. chest; 8. abdomen; and 9. shoulder.

### DETAILED DESCRIPTION OF THE EMBODIMENTS

### Embodiment 1

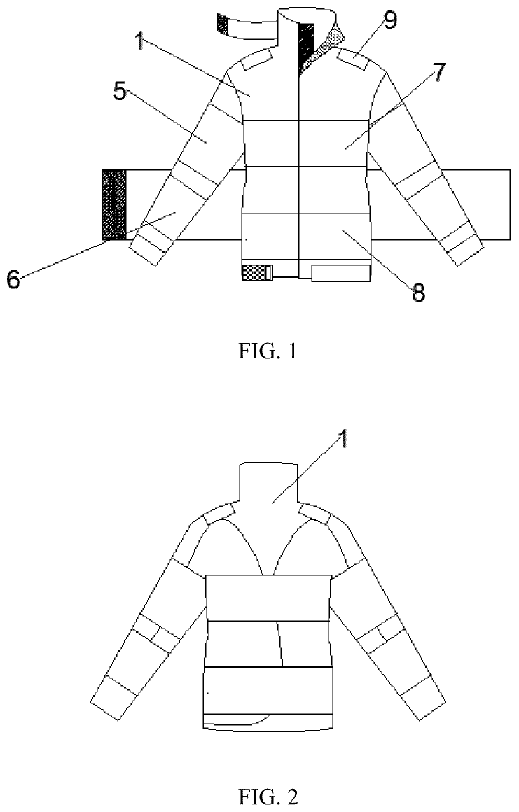

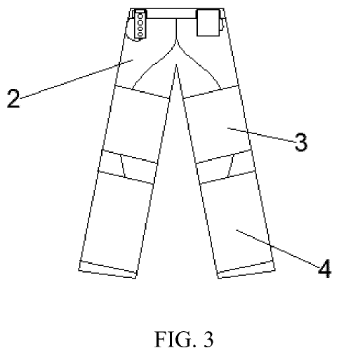

Referring to FIGS. 1-3, an ergonomic magnetic thermal garment includes a jacket 1 and a pair of pants 2, wherein an elastic adjustment piece is arranged at a middle opening of the jacket 1 to adapt to different body shapes;
both the jacket 1 and the pants 2 are provided with magnetic thermal devices; the magnetic thermal devices include a magnetic control mechanism, an electric heating mechanism, and a control mechanism;
the electric heating mechanism is arranged on an inner lining of the jacket 1 and the pants 2 and is connected to the control mechanism;
the magnetic control mechanism is arranged on an outer lining of the jacket 1 and the pants 2 and is connected to the control mechanism;
the control mechanism is also connected to an external power supply mechanism for providing a power supply; and
neck, shoulders 9, back, chest 7, abdomen 8, waist, upper arms 5 and forearms 6 of the jacket, as well as thighs 3, calves 4 and knees of the pants 2, are all detachably connected with fixing accessories for providing mechanical support.

In this embodiment, the fixing accessory is a fixing strap, one side of which is at least partially provided with a hook surface of a hook-and-loop fastener, and an entire other side is provided with a loop surface of the hook-and-loop fastener.

In this embodiment, the external power supply mechanism is a detachable external battery, specifically a power bank.

In this embodiment, the control mechanism includes a controller for controlling the operation of the electric heating mechanism and the magnetic heating mechanism; both the jacket and the pants are provided with fixing parts for installing the external power supply mechanism and the controller.

Preferably, the electric heating mechanism includes electric heating plates, and the electric heating plates are arranged on the inner linings of the jacket 1 and the pants 2 and connected to the control mechanism.

Preferably, the electric heating plate is composed of a plurality of small electric heating plates connected in parallel through a first flexible wire; and the plurality of electric heating plates are distributed on the neck, shoulders 9, back, chest 7, abdomen 8, waist, upper arms 5 and forearms 6 of the jacket, as well as the thighs 3, calves 4, knees and buttocks of the pants.

In this embodiment, since the small electric heating plates are connected in parallel, the control mechanism can control the heat generation of the small electric heating plates in different areas; and there are no small electric heating plates in the perineum area to avoid damage to organs such as the testicles.

Preferably, a temperature of the small electric heating plate is 40-55° C.

Preferably, the magnetic control mechanism includes a plurality of coil assemblies, the coil assemblies are arranged on neck, shoulders 9, back, chest 7, abdomen 8, waist, upper arms 5 and forearms 6 of the jacket, as well as an outer lining of thighs 3, calves 4, knees and buttocks of the pants, and are connected in parallel through a second flexible wire, the coil assemblies are connected to the control mechanism and generate an electromagnetic field through the control mechanism.

In this embodiment, since the coil assemblies are connected in parallel, the control mechanism can control the coil assemblies in different areas to generate electromagnetic fields; and there is no coil assembly in the perineum area to avoid damage to organs such as the testicles.

Preferably, the control mechanism regulates a release position and pulse parameters of the electromagnetic field; and the electromagnetic field is composed of a pulse waveform with a frequency of 15 Hz.

In this embodiment, the specific parameters of the pulse waveform are: a pulse width of 5 ms, a pulse duration of 0.2 ms; a pulse wait of 0.02 ms, a burst wait of 60 ms, a pulse rise time of 0.3 μs, and a pulse fall time of 2 μs.

Preferably, the jacket 1 and the pants 2 are designed according to curves of a human body.

Preferably, an inner lining of the jacket 1 and the pants 2 are made of a heat-resistant insulating material.

In this embodiment, both the jacket 1 and the pants 2 are made of elastic, skin-friendly thermal insulation materials, so that the magnetic thermal garment fits tightly.

Preferably, the elastic adjustment piece is a hook-and-loop fastener, one of hook or loop portions of the hook-and-loop fastener is located on an outer surface of the garment, a portion matched to the hook or loop portions is located on an inner lining of the garment, a size of the hook-and-loop fastener located on the outer surface of the garment is larger than a size of the hook-and-loop fastener located on the inner lining of the garment.

During specific implementation, the control mechanism can control the small electric heating plates and the coil assemblies in the area according to a requirements to generate magnetic heat, which can be used for local thermal magnetic treatment. The power supply is provided by an external power supply mechanism, which can ensure that the magnetic thermal garment is only connected to the power supply when in use, so that safety is ensured, and thermal magnetic treatment for more than 1 hour can be achieved.

The above description is merely preferred embodiments of the present invention, and is not intended to limit the technical scope of the present invention. As such, any minor amendments, equivalent changes and modifications made to the above embodiments according to the technical spirit of the present invention shall fall within the scope of the technical schemes of the present invention.

What is claimed is:

1. An ergonomic magnetic thermal garment, comprising a jacket and a pair of pants, wherein an elastic adjustment piece is arranged at a middle opening of the jacket to adapt to different body shapes;
both the jacket and the pair of pants are provided with magnetic thermal devices; the magnetic thermal devices comprise a magnetic control mechanism, an electric heating mechanism, and a control mechanism;
the electric heating mechanism is arranged on an inner lining of the jacket and the pair of pants and is connected to the control mechanism;
the magnetic control mechanism is arranged on an outer lining of the jacket and the pair of pants and is connected to the control mechanism;
the control mechanism is further connected to an external power supply mechanism for providing a power supply; and
neck, shoulders, back, chest, abdomen, waist, upper arms and forearms of the jacket, as well as thighs, calves and knees of the pair of pants, are all detachably connected with fixing accessories for providing mechanical support.

2. The ergonomic magnetic thermal garment according to claim 1, wherein the electric heating mechanism comprises electric heating plates, the electric heating plates are arranged on the inner linings of the jacket and the pair of pants and connected to the control mechanism.

3. The ergonomic magnetic thermal garment according to claim 2, wherein the electric heating plate is composed of a plurality of small electric heating plates connected in parallel through a first flexible wire; and the plurality of small electric heating plates are distributed on the neck, shoulders, back, chest, abdomen, waist, upper arms and forearms of the jacket, as well as the thighs, calves, knees and buttocks of the pair of pants.

4. The ergonomic magnetic thermal garment according to claim 3, wherein a temperature of each of the plurality of small electric heating plates is 40-55° C.

5. The ergonomic magnetic thermal garment according to claim 1, wherein the magnetic control mechanism comprises a plurality of coil assemblies, the plurality of coil assemblies are arranged on the neck, shoulders, back, chest, abdomen, waist, upper arms and forearms of the jacket, as well as an outer lining of thighs, calves, knees and buttocks of the pair of pants, and are connected in parallel through a second flexible wire, the plurality of coil assemblies are connected to the control mechanism and generate an electromagnetic field through the control mechanism.

6. The ergonomic magnetic thermal garment according to claim 5, wherein the control mechanism regulates a release position and pulse parameters of the electromagnetic field; and
the electromagnetic field is composed of a pulse waveform with a frequency of 15 Hz.

7. The ergonomic magnetic thermal garment according to claim 1, wherein the jacket and the pair of pants are designed according to curves of a human body.

8. The ergonomic magnetic thermal garment according to claim 1, wherein an inner lining of the jacket and the pair of pants are made of a heat-resistant insulating material.

9. The ergonomic magnetic thermal garment according to claim 1, wherein the elastic adjustment piece is a hook-and-loop fastener, one of hook or loop portions of the hook-and-loop fastener is located on an outer surface of the ergonomic magnetic thermal garment, a portion matched to the hook or loop portions is located on an inner lining of the ergonomic magnetic thermal garment, a size of the hook-and-loop fastener located on the outer surface of the ergonomic magnetic thermal garment is larger than a size of the hook-and-loop fastener located on the inner lining of the ergonomic magnetic thermal garment.
